(19) United States
(12) Patent Application Publication
(10) Pub. No.: US 2012/0201869 A1
(43) Pub. Date: Aug. 9, 2012

(54) MARINE BACTERIAL SUBSTANCES, MEDICAL DEVICES, AND METHODS FOR BIOFILM INHIBITION

(76) Inventor: Cynthia K. Burzell, Oceanside, CA (US)

(21) Appl. No.: 13/501,842

(22) PCT Filed: Oct. 14, 2010
(86) PCT No.: PCT/US10/52749

§ 371 Date: Apr. 13, 2012

Related U.S. Application Data
(60) Provisional application No. 61/251,642, filed on Oct. 14, 2009.

(51) Int. Cl. A61K 9/00; A61K 35/74; A61Q 11/00; A01P 1/00; A61P 31/04; A01N 63/02; A61K 8/99

(52) U.S. Cl. 424/400; 424/780; 424/49

(57) ABSTRACT

Disclosed herein are marine bacterial substances, methods, and medical devices that inhibit biofilm growth and/or formation. Substances of the present disclosure are products or byproducts of P3-2 (ATCC PTA-6763), P4-4 (ATCC PTA-6682), P5-2 (ATCC PTA-6764), or P6-6 (ATCC PTA-6766) marine bacterial isolates.

### RELATED APPLICATIONS

This application claims the benefit of U.S. Provisional Application No. 61/251,642 filed October 14, 2009 and incorporates the entirety thereof herein.

### FIELD OF DISCLOSURE

Disclosed herein are marine bacterial substances, medical devices, and methods that inhibit biofilm formation.

### BACKGROUND OF DISCLOSURE

Microbes such as without limitation, Staphylococcus aureus (“S. aureus”), can adhere to surfaces and form biofilms in healthy and immunocompromised hosts. Biofilms, complex microbial communities enclosed in a polymer matrix, are ubiquitous in both probiotic and pathogenic human processes. According to the US National Institute of Health, biofilms are involved in more than 80% of microbial infections.

Biofilms, including, but not limited, to those formed by S. aureus, Staphylococcus epidermidis (“S. epidermidis”) and Pseudomonas aeruginosa (“P. aeruginosa”), can infect nearly every organ system in the body whether associated with indwelling medical devices (e.g., catheter and prostheses infections) or tissues (e.g., chronic wound infections, cystic fibrosis, and endocarditis). Biofilm infections, including S. aureus biofilm infections, are often associated with indwelling medical devices, of which vascular catheter infections pose the greatest risk (Ehrlich et al., (2004) Microbial Biofilms, Ghannoum and O'Toole (Eds). ASM Press, Washington, D.C. pp. 332-358). Central venous catheters (“CVCs”) are the leading cause of nosocomial infections (Maki, (1992) Hospital Infections. Bennett and Brachman (Eds.). 3rd ed. Little, Brown & Co., Boston, Mass., pp. 849-898). The most frequent life-threatening complications associated with CVCs are septicemia, sepsis, vascular occlusion, and abscess formation (Donlan, (2001) Emerg Infect Dis 7: 277-281) which result in an increase in hospital duration, costs, and patient morbidity.

Bacteria form biofilms that are recalcitrant to traditional antibiotic treatments. The dose of antibiotics effective to disrupt a biofilm is approximately 1000× the concentrations that are effective against planktonic bacteria (Desrosiers et al., (2007) Am J Rhinol 21: 149-153). In addition, the close proximity of bacteria in a biofilm increases the incidence of horizontal gene transfer (Li et al., (2001) J Bacteriol 183: 897-908) and acquisition of virulence gene clusters, which not only confer multi-drug resistance, but also can make bacteria more virulent. Multi-drug resistance continues to be a major public health threat especially with S. aureus.

The current treatments for biofilm infections are removal of infected tissue, removal of indwelling medical devices, and/or largely unsuccessful antibiotic therapy. Tissue and device removal are the most effective treatments, however, such treatments can delay healing, damage healthy tissue, and/or prevent critical treatment. Considerable work has been undertaken to investigate alternative treatments and prevention. The successful reduction of colonization by devices coated with antimicrobial agents and antiseptics has been controversial (Kamal et al., (1991) JAMA 265: 2364-2368; Maki et al., (1991) Lancet 338: 339-343). The use of antimicrobial agents (e.g., cephalosporins) may be considered a selective pressure encouraging the emergence of resistant organisms, including methicillin-resistant S. aureus (MRSA) and vancomycin-resistant enterococci (VRE). Ionic antimicrobial metals (i.e., platinum and silver) are being used in catheters to prevent catheter-related bloodstream infections. Silver antibiofilm products, however, select for heavy metal resistant bacteria and may select for antibiotic-resistant bacteria (Davis et al., (2005) Oral Microbiol and Immunol 20: 191-194). The significant medical importance of biofilm infections and the increasing and immediate need for innovative biofilm-inhibiting systems and methods is evident. Accordingly, there is room for improvement in current methods of inhibiting biofilm growth and formation and resulting biofilm infections.

### SUMMARY OF DISCLOSURE

Embodiments disclosed herein include substances produced by marine bacterial isolates and medical devices and methods using the substances to inhibit biofilm growth or formation. The substances of the present disclosure decrease biofilm growth or formation without killing or substantially killing a majority of the bacteria that form the biofilm. Because the substances are non-bactericidal, they allow the biofilm-forming bacteria to remain in their planktonic form so that current antibiotics and immune responses can clear the infection and resistance to the substances is reduced. In certain embodiments the substances are metabolic products (“metabolites”) produced by marine bacteria isolates.

In particular, some embodiments comprise a substance of a marine bacterial isolate, wherein the marine bacterial isolate is P3-2 (ATCC PTA-6763), P4-4 (ATCC PTA-6682), P5-2 (ATCC PTA-6764), P6-5 (ATCC PTA-6765), or P6-6 (ATCC PTA-6766), and wherein the substance inhibits growth or formation of a biofilm.

In some embodiments the substance is a product or byproduct of the exponential growth phase of the marine bacterial isolate. But in other embodiments, it is a product or byproduct of the stationary growth phase of the marine bacterial isolate. In certain embodiments the substance is a metabolite of the exponential or stationary growth phases. In addition, in one embodiment the substance is an ether extract.

In one embodiment, the biofilm inhibited by the substance is formed by S. aureus. In another embodiment the biofilm inhibited by the substance is formed by S. epidermidis, and in yet another embodiment the biofilm inhibited by the substance is formed by P. aeruginosa.

Some embodiments include a method of inhibiting growth of a biofilm comprising selecting a marine bacterial isolate of P3-2 (ATCC PTA-6763), P4-4 (ATCC PTA-6682), P5-2 (ATCC PTA-6764), P6-5 (ATCC PTA-6765), or P6-6 (ATCC PTA-6766); extracting a substance from a culture of the marine bacterial isolate; and, applying the substance to a biofilm. In one embodiment, the substance extracted from a culture is a metabolite.

In some embodiments, the method is used with medical devices.

In one embodiment the extracting step of the disclosed method is completed during the exponential growth phase of the marine bacterial isolate. In another embodiment, the extracting step is completed during the stationary growth phase of the isolate. And, in yet another embodiment, the extracting step comprises shaking supernatant of the culture with an equal aliquot of diethyl-ether and after a period of time shaking the solution of diethyl-ether and supernatant with Tris-phosphate EDTA.

In certain embodiments, the biofilm can be pathogenic, for example it can be formed by, without limitation, S. aureus, S. epidermidis, or P. aeruginosa.

One embodiment includes a method of producing a medical device comprising coating at least a portion of a medical device with an antibiofilm composition, wherein the antibiofilm composition comprises a substance produced by a marine bacterial isolate. In one embodiment the marine bacterial isolate is P3-2 (ATCC PTA-6763), P4-4 (ATCC PTA-6682), P5-2 (ATCC PTA-6764), P6-5 (ATCC PTA-6765), or P6-6 (ATCC PTA-6766). Another embodiment of a method of producing a medical device comprises incorporating a substance produced by a marine bacterial isolate of P3-2 (ATCC PTA-6763), P4-4 (ATCC PTA-6682), P5-2 (ATCC PTA-6764), P6-5 (ATCC PTA-6765), or P6-6 (ATCC PTA-6766) into a wash, a nasal spray, a topical gel, toothpaste, mouth wash, or eye drops. In some such embodiments, the substance incorporated into the medical device is a metabolite of the stationary or exponential growth phase of a marine bacterial isolate.

An embodiment of a medical device of the present disclosure comprises an antibiofilm composition, wherein the antibiofilm composition comprises a substance produced by an isolate of P3-2 (ATCC PTA-6763), P4-4 (ATCC PTA-6682), P5-2 (ATCC PTA-6764), P6-5 (ATCC PTA-6765), or P6-6 (ATCC PTA-6766). In one such embodiment, the antibiofilm composition is a coating layer on at least a portion of the outer surface of the medical device.

In some embodiments of the disclosed medical devices, the substance is a product or byproduct of the exponential growth phase of the marine bacterial isolate. In other embodiments, the substance is a product or byproduct of the stationary growth phase of the isolate. In one embodiment, the substance is a metabolite of either the exponential or stationary growth phase.

Finally, in certain embodiments of the medical device, the antibiofilm composition inhibits growth or formation of a biofilm by S. aureus, S. epidermidis, or P. aeruginosa.

### BRIEF DESCRIPTION OF THE FIGURES

FIGS. 1A-1F are confocal microscopic images of a S. aureus biofilm.

FIG. 2 is a graph illustrating 24 hour growth of S. aureus in the presence of P3-2 supernatant and a negative control.

### DETAILED DESCRIPTION OF THE DISCLOSURE

The embodiments of this disclosure use, wholly or partially, substantially non-bactericidal substances, including without limitation metabolites, to inhibit biofilm growth or formation.

The disclosed substances can inhibit biofilm growth or formation without killing or substantially killing the majority of biofilm bacteria. For example, P3-2, P4-4, P5-2, and P6-6 produce substances that are non-toxic. The use of substantially non-bactericidal substances in medical devices allows for the bacteria to remain in their planktonic form so that current antibiotics and immune responses can clear the infection. In addition, substances that prevent biofilm formation without substantially killing bacteria can reduce the incidence of resistance to those substances. An effective strategy that reduces the risk of biofilm infections will generate health benefits by reducing the incidence of illness, reducing cost of care, and reducing the number of associated deaths. The substances disclosed herein can inhibit biofilm growth or formation by S. aureus, S. epidermidis, P. aeruginosa, and other biofilm-forming bacteria without substantial killing of the majority of bacteria (i.e., they are non-bactericidal).

“Substance” or “marine bacterial substance” as used interchangeably herein refers to a product or a byproduct produced by marine bacterial isolates or as a result of the interaction between marine bacterial isolates and their environments, which can inhibit the formation or growth of biofilms. For example, a substance can include, but is not limited to, metabolites of marine bacterial isolates. Substances include, without limitation, products and byproducts of P3-2 (ATCC PTA-6763; NCIMB 41696), P4-4 (ATCC PTA-6682; NCIMB 41694), P5-2 (ATCC PTA-6764; NCIMB 41695), P6-5 (ATCC PTA-6765), or P6-6 (ATCC PTA-6766). The above “PTA” designations are for deposits at the American Type Culture Collection (“ATCC”) patent depository, and the “NCIMB” designations are for deposits at the National Collection of Industrial, Food, and Marine Bacteria patent depository.

“Medical devices” as used herein includes any device, solution, or antibiofilm composition that effectuates or is intended to effectuate a medical treatment. For example, the term medical devices includes, without limitation, indwelling medical devices, CVCs, contact lenses, urinary catheters, stents, peritoneal dialysis catheters, prosthetic joints, pacemakers, mechanical heart valves, endotracheal tubes, intrauterine devices, tympanostomy tubes, drug delivery devices, implants, artificial organs, and voice prostheses. The term medical devices also includes, without limitation, solutions and compositions used in nasal sprays, eye drops, mouth washes, toothpastes, topical gels, ointments, or washes. In addition, “medical devices” as used herein also includes surfaces, garments, and materials that come into contact with patients during medical treatments or procedures. For example, without limitation, the term medical devices includes scrubs, robes, clothes, gauze, operating tables, beds, table covers, sheets, and other clinically relevant surfaces.

“Antibiofilm compositions” are any solid, liquid, or gas phase that includes one or more marine bacterial substance and one or more other components necessary or helpful to inhibiting biofilm growth or formation or another intended purpose (such as, in one example, a secondary medical treatment). For example, antibiofilm compositions can include, without limitation, sprays, solutions, mists, solids, or vapors. Other components incorporated into antibiofilm compositions, can include, without limitation, buffers, solvents, preservatives, antibiotics, antifungal agents, antihistamines, anti-inflammatory agents, bonding or binding agents, neutralization agents, or precipitating agents.

Polyphasic taxonomy was performed to characterize isolated marine bacteria. Isolates P3-2 (ATCC PTA-6763), P4-4 (ATCC PTA-6682), and P5-2 (ATCC PTA-6764) are novel species belonging respectively to the Aerococcus genus, termed A. piscidermidis; Psychrobacter genus, termed P. piscidermidis; and Erythrobacter genus, termed E. piscidermidis. It is proposed that isolate P6-6 (ATCC PTA-6766) represents a novel genus, termed Brunonia piscidermidis.

Substances, including but not limited to metabolites, from novel marine bacterial isolates inhibit biofilm growth and/or formation. For example, P3-2, P4-4, P5-2, P6-5, and P6-6 can inhibit S. aureus (ATCC 25923 and ATCC 12600), S. epidermidis (ATCC 12228), and P. aeruginosa (ATCC 27853) biofilm growth or formation. In some embodiments, S. aureus biofilm growth or formation can be inhibited up to 61% (p<0.01); S. epidermidis biofilm growth or formation can be inhibited up to 35% (p<0.01); and P. aeruginosa can be inhibited up to 10% (p<0.01) by one or more substance of the present disclosure In other embodiments, S. aureus biofilm growth or formation can be inhibited up to 3%, 5%, 11% or 48% by one or more substance of the present disclosure. In certain embodiments, S. epidermidis biofilm growth or formation can be inhibited up to 9%, 13%, 14%, 16%, 29%, or 31% by one or more substance of the present disclosure. Tables 1 and 2 indicate exemplary inhibition of biofilm growth and/or formation by the disclosed substances.

Some substances of the present disclosure are products or byproducts of the exponential growth phase of marine bacterial isolates. Other substances are products or byproducts of the stationary growth phase of marine bacterial isolates. In some embodiments, substances are metabolites. As known by those of ordinary skill in the art, substances produced in the exponential growth phase, including primary metabolites, are generally responsible for growth and reproduction, whereas substances produced in the stationary growth phase, including secondary metabolites, are generally responsible for defense.

In certain embodiments, the marine bacterial substances of isolates of P3-2, P4-4, P-5-2, P6-5, or P6-6 are used in an antibiofilm composition and/or on medical devices. Such antibiofilm compositions can be used as a coating for medical devices or impregnated or otherwise included in medical devices to inhibit the growth or formation of biofilms, thereby reducing the incidence of infection. In another aspect, the substances or an antibiofilm composition can be used in medical devices, such as without limitation, a wash for wounds to inhibit biofilm growth or formation thereby reducing the incidence of wound infections. In other embodiments, an antibiofilm composition of the present description can be used in a nasal spray to inhibit biofilm growth or formation, thereby preventing and/or treating infections, including without limitation, nasal infections. Alternatively, substances or an antibiofilm composition can be used in eye drops to inhibit biofilm growth or formation, thereby preventing and/or treating eye infections.

The concentration of the disclosed substances used in an antibiofilm composition can be up to 5%, up to 15%, up to 30%, up to 60%, or up to 99.9% of the antibiofilm composition. In embodiments where the antibiofilm composition is used as a coating, such a coating may cover up to 10%, up to 25%, up 50%, or up to 100% of the medical device.

Coatings can be applied to the surface of a medical device. Processes of coating medical devices can include, but are not limited, to chemical vapor deposition, physical vapor deposition, chemical and electrochemical techniques, spraying, dip-coating, painting, applying a polymer or powder, or spin-coating. These techniques are known to those of ordinary skill in the art.

In some embodiments where the antibiofilm composition is used as a coating or impregnated or otherwise included in a medical device, in addition to the substance, the antibiofilm composition can comprise at least one binding module to specifically bind the antibiofilm composition to the medical device such as, without limitation, a lectin. In other embodiments, the disclosed substance can be disposed within (chemically coupled or entrapped) in a biodegradable polymer. In certain of such embodiments, the polymer can degrade over a period of days, weeks, or months. In one embodiment, a lectin can be disposed within the polymer as a binding agent. In another embodiment, the substance can be disposed within (chemically coupled or entrapped) in a water-soluble polymer. Either naturally-occurring or synthetic polymers—including without limitation, polyvinyl alcohol, poly(ethylene glycol), poly(lactic-co-glycolic acid), polysaccharides such as dextran or ficoll, or proteins such as polylysine—can be used in antibiofilm compositions. In addition, in one embodiment, an antibiotic (for example, without limitation, streptomycin, penicillin, ciprofloxacin, gentamycin, methicillin, vancomycin, or lincomycin) can be included along with the substance in the antibiofilm composition.

In some embodiments, the substances of the present disclosure can be concentrated and/or dried. Substances can be dried through the process of freeze drying (lyophilization) or evaporation. Dried marine bacterial substance can be re-suspended in different solutions, including antibiofilm compositions, at different concentrations. For example, it can be added to an existing eye drop solution (with or without dilution) at different concentrations, to determine which concentration is the most effective.

Certain substances lose their chemical structure or function when completely dried. In this circumstance, the substances can be concentrated by evaporation or filter-concentrated by centrifugation.

### Example 1

### Inhibition of Biofilm Formation Using Marine Bacterial Substances

Isolation of substances: Marine bacterial isolates P4-4, P5-2, and P6-6 can be cultured on Artificial Sea Water (ASW) media. ASW broth contained (g/l) of solution: NaCl 21.10, KCl 0.58, CaCl₂×2H₂O 1.20, MgCl₂×6H₂O 4.73, NaHCO₃ 0.08, MgSO₄×7H₂O 2.63, yeast extract 10.00, malt extract 4.00, and glucose 4.00, and agar 15.00. Plates were incubated at 29° C. Unless otherwise stated, marine isolate P3-2 is cultivated on Trypticase Soy Broth (TSB) (Difco) plus NaCl (30.0 g/L) and yeast extract (3.0 g/L) at 29° C.

The marine bacterial isolates were grown in flasks half filled with appropriate media. Cultures were incubated in a shaking incubator at 29° C. at 180 rpm. The supernatants were collected during the exponential and/or stationary stages of growth, determined by growth curve analysis. The cells were separated from the supernatant by centrifugation at 5,000 rpm and 10° C. for 5 minutes in 50 ml centrifuge tubes. Centrifugation was repeated three times. Supernatants were filtered using a 0.22 um filter.

Ether extraction: Crude extracts were obtained from the supernatant culture medium. After supernatant pH was reduced to 2.0, it was shaken with an equal aliquot of diethyl-ether for 5 minutes. After 10 minutes standing, the ether portion was shaken for 5 minutes with Tris-phosphate EDTA buffer (Sigma) at pH 8.0 to re-extract the substances into the water. The aqueous portion was termed “ether extract.” Negative controls using sterile ASW broth was treated in the same way as the extracts.

Biofilm assay: Cultures of pathogenic biofilm forming S. aureus (ATCC 25923), S. epidermidis (ATCC 12228), and P. aeruginosa (ATCC 27853) were grown in TSB at 37° C. Biofilm assays were performed in TSB+0.25% glucose. Overnight cultures were diluted 1:100 in fresh culture media and grown (150 μl) in 96 well microplates in presence or absence of a 50 μl metabolite or control for 24 or hours at 37° C. (Merritt et al., (2005) Current Protocols in Microbiology. New Jersey: John Wiley and Sons. pp. 1-17). The positive controls were penicillin or vancomycin with final concentrations of 1000 μg/ml—an antibiotic dose that would be toxic to humans.

The planktonic bacteria were removed by washing with sterile distilled water and stained by crystal violet (0.1%) for 10 minutes. The plates were washed to remove unbound stain and air-dried at room temperature. The staining solution was eluted from the biofilm by 95% ethanol or 33% glacial acetic acid at 37° C. for 10 minutes. Plates were read in a microtiter plate reader at 490 nm. The percent biofilm reduction was determined for each sample and a Student t-Test (2 tailed, equal variance) was used to determine if the percent reduction was statistically significant.

Percent biofilm reduction was determined for P3-2, P4-4, P5-2, and P6-6 substances using these methods by comparison to negative controls. S. aureus, S. epidermidis, and P. aeruginosa biofilm formed in the negative control, but was inhibited in the presence of marine bacterial substances. Results are indicated in Tables 1 and 2.

TABLE 1
Table 1. Antibiofilm activity of exponential phase metabolites
from selected marine bacterial isolates against 24 hour S. aureus
(“SA”) and/or S. epidermidis (“SE”) biofilms
(n = 36) compared to negative controls (n = 24).

| | SA (ATCC 25923) | SE (ATCC 12228) |
|---|---|---|
| Sample | Biofilm Inhibition (%) | Biofilm Inhibition (%) |
| P5-2 | | |
| Ether Extract | — | 16.45 ** |
| Supernatant | 10.62 ** | — |
| P3-2 | | |
| Supernatant | 48.16 ** | 35.41 ** |
| P6-6 | | |
| Ether Extract | — | 29.43 ** |
| Supernatant | 10.68 ** | — |

** indicates a p value < 0.01.

TABLE 2
Table 2. Antibiofilm activity of stationary phase supernatants
from selected marine isolates against 24 hour S. aureus (“SA”),
S. epidermidis (“SE”), and/or P. aeruginosa
(“PA”)_biofilms (n = 36) compared to negative controls (n = 24).

| | SA (ATCC | SA (ATCC | SE (ATCC | PA (ATCC |
|---|---|---|---|---|
| | 25923) | 12600) | 12228) | 27853) |
| Sam- | Biofilm | Biofilm | Biofilm | Biofilm |
| ple | Inhibition (%) | Inhibition (%) | Inhibition (%) | Inhibition (%) |
| P4-4 | — | — | 9.06 * | 9.65 ** |
| P5-2 | 5.11 ** | — | 12.96 ** | 9.30 ** |
| P3-2 | 61.11 ** | 32.35 ** | 30.46 ** | — |
| P6-6 | 3.76 * | 36.30 ** | 14.09 ** | 9.71 ** |

* indicates a p value < 0.05;
** indicates a p value < 0.01.

Stationary phase P3-2 supernatant resulted in the highest S. aureus antibiofilm activity at 61% (p<0.01) according to the biofilm assay. This indicates that P3-2 is the most effective against S. aureus biofilm formation. Exponential phase P3-2 supernatant also inhibited S. arueus biofilm growth and formation up to 48% (p<0.01) compared to the negative control. Similarly, exponential phase P3-2 supernatant resulted in the highest S. epidermidis antibiofilm activity at 35% (p<0.01) compared to the negative control.

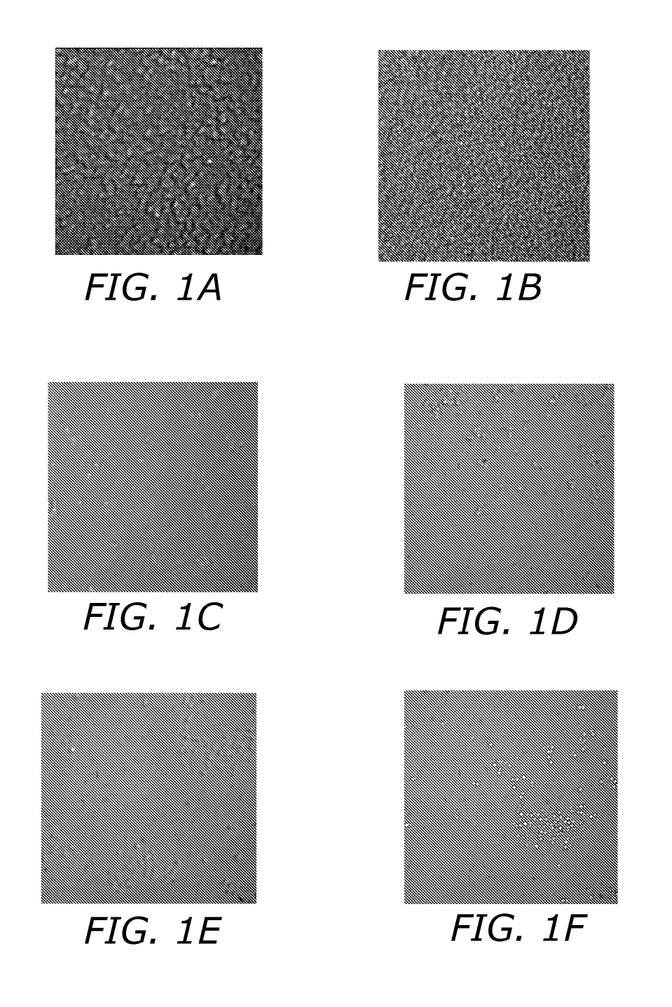

FIG. 1 illustrates confocal microscopic images of 24 hour S. aureus biofilm (stained with congo red) in the negative control (FIGS. 1A and 1B), P3-2 stationary phase supernatant (FIGS. 1C and 1D), and positive control (FIGS. 1E and 1F). Biofilm grew in the negative control as illustrated in FIGS. 1A and 1B, but was inhibited by a stationary phase P3-2 supernatant as seen in FIGS. 1C and 1D. Biofilm was also inhibited by the high concentration antibiotic in the positive control as shown in FIGS. 1E and 1F. FIGS. 1C-1F show the small patches of thin, loosely associated biofilms, in contrast to the dense biofilm of the negative control shown in FIGS. 1A and 1B.

A biofilm assay of isolate P3-2 supernatant against 48 hour S. aureus biofilm formation indicated that P3-2 antibiofilm activity was sustained for 48 hours.

Microplate assays were repeated using concentrated proteins (3 kDa and 10 kDa cut off) and 10 kDa rejected filtrate to determine the nature of the substance(s). Concentrated proteins did not inhibit S. aureus biofilm formation. P3-2 supernatant 10 kDa rejected filtrate (n=23) significantly inhibited S. aureus biofilm formation by 64.03% (p<0.01) when compared to a negative control (n=24). This indicates that isolate P3-2 S. aureus antibiofilm activity was due to a small molecule or peptide smaller than 10 kDa.

### Example 2

### Mutagenicity of Marine Bacterial Substances

Genotoxicity was determined for P3-2 exponential and stationary phase supernatants according to the Ames test (Ames et al., (1973) Proc Nat Acad Sci USA 70: 782-786.) with three auxotrophic Salmonella enterica strains (previously S. typhimurium) (ATCC 29629, ATCC 29630, ATCC 29631) obtained from the ATCC (Manassas, Va.). The results were considered positive if the number of revertants were at least twice as high as the negative control. P3-2 exponential (n=2) and stationary phase (n=4) supernatants reverted the three S. enterica mutant strains the same as or less than the negative control (n=2) (Table 3). Therefore, the Ames test results were negative. P3-2 exponential and stationary phase supernatants were determined to be free from genotoxins according to the Ames test.

TABLE 3
Ames test results for P32 exponential
and stationary phase supernatants.

| | Average number of revertants ± SD | | |
|---|---|---|---|
| Experimental | S. enterica | S. enterica | S. enterica |
| condition | ATCC 29629 | ATCC 29630 | ATCC 29631 |
| Positive Control | 38 ± 4 | 101 ± 21 | 246 ± 77 |
| Negative Control | 5 ± 1 | 4 ± 1 | 10 ± 8 |
| P3-2 EP supernatant | 5 ± 4 | 3 ± 1 | 5 ± 3 |
| P3-2 SP supernatant | 3 ± 2 | 4 ± 2 | 6 ± 4 |

SD, standard deviation; EP, exponential phase; SP, stationary phase.

### Example 3

### Non-Killing Nature of Marine Bacterial Substances

Antibacterial Activity: To determine if the substances have antibacterial properties the Kirby Bauer Method was used (Bauer et al., (1966) Am J Clin Pathol 45: 493-6.). Extract/supernatant (10 μl or 20 μl) was used to inoculate sterile 6 mm disks (Difco). Disks containing 20 μl were prepared first by adding 10 μl, allowing disk to dry and then adding an additional 10 μl. Zones were measured after 24 hours of incubation at 37° C. The experiments were performed in triplicate. Penicillin (IU/IE/UI) was used as a positive control. Samples were considered to contain antibacterial activity if the diameter of the zone of clearance was within the sensitive range for penicillin (which is greater than or equal to 29 mm). None of the marine bacterial supernatants or extracts resulted in antibacterial activity against S. aureus, S. epidermidis, or P. aeruginosa.

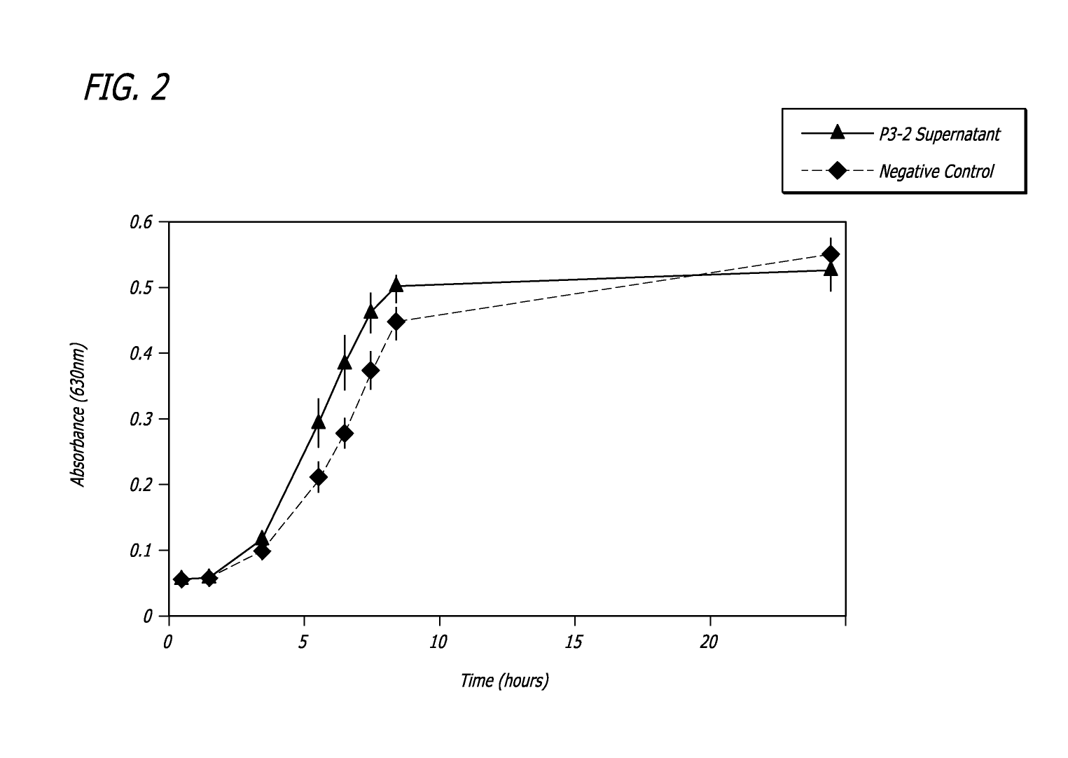

Growth Curve Analysis: The antibiofilm compositions and substances of the present description, for example without limitation P3-2, are novel and inhibit biofilm formation without inhibiting bacterial growth, which is emphasized in FIG. 2. FIG. 2 is a graph illustrating 24 hour growth of S. aureus in the presence of P3-2 supernatant (depicted by the solid line) and a negative control (depicted by the dotted line). Error bars represent standard deviation values for each mean displayed on the graph. As shown in FIG. 2, the P3-2 supernatant (n=20) did not inhibit S. aureus (ATCC 25923) growth compared to negative control (n=20).

### Example 4

### Novelty of Substance of P3-2 Isolate

Structural characterization of P3-2 stationary phase supernatant, P4-4 exponential phase supernatant, P5-2 stationary phase ether extract, and P6-6 exponential phase ether extract was performed using Gas Chromatography Mass Spectrometry (GC-MS). Mass spectometry data was presented along with a computer Library Search Report. Matches >95% were not identified in the National Institute of Standards and Technology (“NIST”) database, suggesting novelty of these substances.

### Example 5

### S. aureus Antibiofilm Activity on Clinically Relevant Surfaces

Adherence of bacteria to medical devices, such as, without limitation, catheters, and host components is a critical step of insertion site infections, abscess formation, cellulitis, vascular occlusions, etc. In order to validate substance antibiofilm activity on catheters and human epithelial cells, a Student t-Test (2 tailed, equal variance) will be used to determine if the percent inhibition of S. aureus bacteria adherence or biofilm formation is statistically significant from a negative control. The results will indicate effectiveness.

Medical device adherence assay: The prevention of S. aureus biofilm formation on polyurethane CVCs will be determined using sterilely-cut pieces of CVCs. The prevention of antibiofilm activity on other clinically relevant surfaces will be determined using sterilely-cut pieces of stainless steel, plastic, wood, vinyl, glass, and cotton. The sterilely-cut pieces of CVCs, stainless steel, plastic, wood, vinyl, glass, and cotton will be placed in the presence of disclosed substances and diluted in an overnight culture of S. aureus. The pieces will be washed, sonicated and vortexed (10 s+10 s) twice in phosphate buffered saline (PBS). An aliquot of the bacterial suspension will be serially diluted and plated for viable count. The results will indicate a decrease in adherence of S. aureus to medical device in presence of marine bacterial substance compared to negative control.

Cell adherence assay: A cell adherence assay to determine S. aureus antibiofilm activity of marine bacterial substances on HaCaT human keratinocytes epithelial cells will be carried out in a microtiter system. Viable count will be used to quantify level of adherence. The results will indicate a decrease in adherence of S. aureus to epithelial cells in presence of marine bacterial substance compared to negative control.

### Example 6

### Use of Marine Bacterial Substance in Toothpaste to Control Dental Plaque and Associated Oral Pathology

A toothpaste is prepared using 10% w/v marine bacterial substance. The toothpaste can be used 2 to 3 times per day. The toothpaste can cause an inhibition of colonizers, rendering the tooth enamel accessible to cleaning by the dentifrice. The marine bacterial substance can prevent the formation of new plaque by inhibiting plaque-forming species. Accordingly, the toothpaste can help prevent or reduce dental plaque and associated oral pathology including but not limited to, dental carries, gingivitis, periodontal diseases, and halitosis.

### Example 7

### Use of Marine Bacterial Substance in Mouthwash to Control Dental Plaque and Associated Oral Pathology

A mouthwash solution is prepared using 10% w/v and 20% w/v marine bacterial substance. Several milliliters of the mouthwash are used to rinse the teeth and gums. The mouthwash can cause an inhibition of colonizers, rendering the tooth enamel accessible to cleaning by the dentifrice. The marine bacterial substance can also prevent the formation of new plaque by inhibiting plaque-forming species. When applied orally, the mouthwash help prevent or reduce dental plaque and associated oral pathology including but not limited to, dental carries, gingivitis, periodontal diseases, and halitosis.

### Example 8

### Use of Marine Bacterial Substance in Topical Gel to Control Skin Infections, Wound Infections, Burns, Acne, and Rosacea

A topical gel is prepared containing component % (by weight): marine bacterial substance 10, ethanol 65, and polyethylene glycol 25. Polyethylene glycol can be substituted with other appropriate carriers. After components are combined, the solution can be set aside for several hours to allow gel to form. The formulation can be combined with or without antibiotics. When applied topically, the gel can help prevent or reduce skin infections, wound infections, burns, acne, and rocacea.

### Example 9

### Use of Marine Bacterial Substance in Wash to Control Skin Infections, Wound Infections, Burns, Acne, and Rosacea

A wash is prepared using marine bacterial substance 20% w/v in normal saline solution (0.9% NaCl). Formulation can be combined with or without antibiotics. When applied topically, the wash can help prevent or reduce skin infections, wound infections, burns, acne, and rocacea.

### Example 10

### Use of Marine Bacterial Substance in Nasal Spray to Control Nasal and Sinus Infections

A nasal spray solution is prepared using marine bacterial substance 20% w/v in normal saline solution (0.9% NaCl). The formulation can be combined with or without antibiotics and/or antihistamines. One spray can deliver 50 μl of nasal spray solution of into the nose. When sprayed into the nose the nasal spray can help prevent or reduce nasal and sinus infections.

### Example 11

### Use of Marine Bacterial Substance in Eye Drops or Contact Lens Solution to Control Eye Infections

An eye drop or contact lens solution is prepared using marine bacterial substance 20% w/v in normal saline solution (0.9% NaCl). The formulation can be combined with or without antibiotics and/or antihistamines. One drop can deliver 50 μl of eye drop solution into the eye. When dropped into the eye the eye drop solution can help prevent or reduce eye infections. When the solution is used on contact lenses the solution can inhibit biofilms from forming on lenses and subsequently reduce the incidence of contact lens related eye infections.

### Example 12

### Medical Device Coating Formulation

Polyvinyl alcohol (PVA) is a copolymer of vinyl alcohol and vinyl acetic acid. A coating is prepared using PVA (50 g/l) and marine bacterial substance (20 g/l). The PVA coating formulation can be stored at room temperate in a covered/sealed container for about 5 days after preparation at ambient temperature, and about 3 months at about 38° C. The PVA coating formulation may normally be used at 38° C.

### Example 13

### Medical Device Cross-Linking Formulation

Chemical cross-linking is the formation of chemical bonds between polymer chains. Cross-linking can increase strength and toughness. Cross-linking solution is prepared using 37% HCl (27 ml/l), 40% glyoxal (25 ml/l), 37% formaldehyde (81 ml/l). Cross-linker is stored at room temperature in a covered container until it is used. The shelf-life is 90 days from the date of preparation.

### Example 14

### Medical Device Coating Method

A catheter is submerged into a coating formulation and spun at 2 rpm for 30 seconds at 38° C. The catheter is withdrawn from the coating formulation at 5-7 mm/second at 5 rpm. The catheter is dried for 10 minutes at 83° F. This step can be repeated to coat catheter multiple times. After the final coat is dried, the catheter is then submerged into the cross-linking formulation at 5 rpm for 40 seconds. The catheter is withdrawn from the cross-linking formulation at 25 mm/sec at 5 rpm. The catheter is dried for 10 minutes at 83° F.

Users in a wide variety of fields, including without limitation medical practitioners, will find that the methods, medical devices, and substances disclosed herein provide many advantages over existing methods of preventing or reducing the occurrence or severity of infections caused by biofilms.

Unless otherwise indicated, all numbers expressing quantities and/or properties used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the exemplary embodiments (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the exemplary embodiments and does not pose a limitation on the scope of the exemplary embodiments otherwise claimed. No language in the specification should be construed as indicating any non-claimed element essential to the practice of the exemplary embodiments.

Groupings of alternative elements or embodiments disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments are described herein, including the best mode known to the inventors for carrying out the exemplary embodiments. Of course, variations on these described embodiments will become apparent to those of ordinary skill in the art upon reading the foregoing description. The inventor expects skilled artisans to employ such variations as appropriate, and the inventors intend for the embodiments to be practiced otherwise than specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

Furthermore, numerous references have been made to patents and printed publications. Each of the above-cited references is individually incorporated herein by reference in their entirety.

Specific embodiments disclosed herein may be further limited in the claims using consisting of or and consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Exemplary embodiments so claimed are inherently or expressly described and enabled herein.

In closing, it is to be understood that the exemplary embodiments disclosed herein are illustrative of the principles of the present disclosure. Other modifications that may be employed are within the scope of the disclosure. Thus, by way of example, but not of limitation, alternative configurations of the present exemplary embodiments may be utilized in accordance with the teachings herein. Accordingly, the present exemplary embodiments are not limited to that precisely as shown and described.

What is claimed is:

1. A substance of a marine bacterial isolate, wherein said marine bacterial isolate is P3-2 (ATCC PTA-6763), P4-4 (ATCC PTA-6682), P5-2 (ATCC PTA-6764), or P6-6 (ATCC PTA-6766), and wherein said substance inhibits growth or formation of a biofilm.

2. A substance of claim 1, wherein said substance is a product or byproduct of the exponential growth phase of said marine bacterial isolate.

3. A substance of claim 1, wherein said substance is a product or byproduct of the stationary growth phase of said marine bacterial isolate.

4. A substance of claim 1, wherein said substance is an ether extract.

5. A substance of claim 1, wherein said biofilm is formed by Staphylococcus aureus (“S. aureus”).

6. A substance of claim 1, wherein said biofilm is formed by Staphylococcus epidermidis (“S. epidermidis”).

7. A substance of claim 1, wherein said biofilm is formed by Pseudomonas aeruginosa (“S. aeruginosa”).

8. A method of producing a medical device comprising incorporating a substance of a marine bacterial isolate into and/or onto said medical device, wherein said marine bacterial isolate is P3-2 (ATCC PTA-6763), P4-4 (ATCC PTA-6682), P5-2 (ATCC PTA-6764), or P6-6 (ATCC PTA-6766) and the substance inhibits growth or formation of a biofilm.

9. A method of claim 8, wherein said incorporating step comprises applying a coating onto said medical device.

10. A method of claim 8, wherein said substance is part of an antibiofilm composition.

11. A method of claim 8, wherein said medical device is a catheter.

12. A method of claim 8, wherein said substance is a product or byproduct of the exponential or stationary growth phase of said marine bacterial isolate.

13. A method of claim 8, wherein said substance inhibits growth or formation of a biofilm by S. aureus, S. epidermidis, or P. aeruginosa.

14. A medical device comprising a substance of a marine bacterial isolate, wherein said marine bacterial isolate is P3-2 (ATCC PTA-6763), P4-4 (ATCC PTA-6682), P5-2 (ATCC PTA-6764), P6-5 (ATCC PTA-6765), or P6-6 (ATCC PTA-6766) and the substance inhibits growth or formation of a biofilm.

15. A medical device of claim 14, wherein said substance is provided as a coating on at least a portion of the outer surface of said medical device.

16. A medical device of claim 14, wherein said substance is part of an antibiofilm composition.

17. A medical device of claim 14, wherein said medical device is a catheter.

18. A medical device of claim 14, wherein said substance is a product or byproduct of the exponential growth phase of said marine bacterial isolate.

19. A medical device of claim 14, wherein said substance is a product or byproduct of the stationary growth phase of said marine bacterial isolate.

20. A medical device of claim 14, wherein said substance inhibits growth or formation of a biofilm by S. aureus, S. epidermidis, or P. aeruginosa.
